(19) United States
(12) Patent Application Publication
(10) Pub. No.: US 2017/0216072 A1
(43) Pub. Date: Aug. 3, 2017

(54) ANKLE STIRRUP

(71) Applicant: Tung-Cheng CHEN, Taichung City (TW)

(72) Inventor: Tung-Cheng CHEN, Taichung City (TW)

(21) Appl. No.: 15/013,295

(22) Filed: Feb. 2, 2016

(51) Int. Cl. A61F 5/01; A61F 5/37

(52) U.S. Cl. CPC A61F 5/0111; A61F 5/37

(57) ABSTRACT

An ankle stirrup has an ankle holder, a positioning unit, and multiple mounting belts. The ankle holder has a leg portion comprising a hole formed through the leg portion, and a foot portion. The positioning unit is mounted in the hole of the ankle holder, and has a body being elongated and having a longitudinal direction and a rotatable portion formed on a front side of the body, inserted through, engaging, and being rotatable relative to the hole. The positioning unit further has a limiting portion detachably mounted in the rotatable portion to keep the rotatable portion from detaching from the hole.

### BACKGROUND OF THE INVENTION

1. Field of the Invention

The present invention relates to a stirrup, and more particularly to an ankle stirrup.

2. Description of Related Art

A conventional ankle stirrup comprises a holder and multiple belts. The belts are mounted on the holder. The holder is formed in an L-shape in section to be applied under an ankle of a user. The belts are mounted around a leg of the user, such that the ankle stirrup is applied for fixing the ankle of the user.

However, when the user lies on a bed, the legs of the user may turn relative to the bed, so a fixing effect for the ankle of the user may be decreased. The conventional ankle stirrup is not suitable to be used while the user is sleeping in bed. Therefore, the conventional ankle stirrup should be improved to resolve the aforementioned problem.

### SUMMARY OF THE INVENTION

The main objective of the present invention is to provide an improved ankle stirrup to resolve the aforementioned problem.

The ankle stirrup comprises an ankle holder, a positioning unit, and multiple mounting belts.

The ankle holder comprises a leg portion comprising a hole formed through the leg portion, and a foot portion.

The positioning unit is mounted in the hole of the ankle holder, and comprises a body being elongated and having a longitudinal direction and a rotatable portion formed on a front side of the body, the rotatable portion inserted through, engaging, and being rotatable relative to the hole. The positioning unit further comprises a limiting portion detachably mounted in the rotatable portion to keep the rotatable portion from detaching from the hole.

The mounting belts are mounted on the ankle holder.

Other objectives, advantages and novel features of the present invention will become more apparent from the following detailed description when taken in conjunction with the accompanying drawings.

### BRIEF DESCRIPTION OF THE DRAWINGS

FIGS. 1 and 2 are perspective views of a preferred embodiment of an ankle stirrup in accordance with the present invention;

FIG. 3 is a side view of the ankle stirrup in FIG. 1;

FIG. 4 is an exploded perspective view of the ankle stirrup in FIG. 1;

FIG. 5 is an enlarged exploded perspective view of the ankle stirrup in FIG. 4; and

FIGS. 6 to 8 are operational views of the ankle stirrup in FIG. 1.

### DETAILED DESCRIPTION OF PREFERRED EMBODIMENT

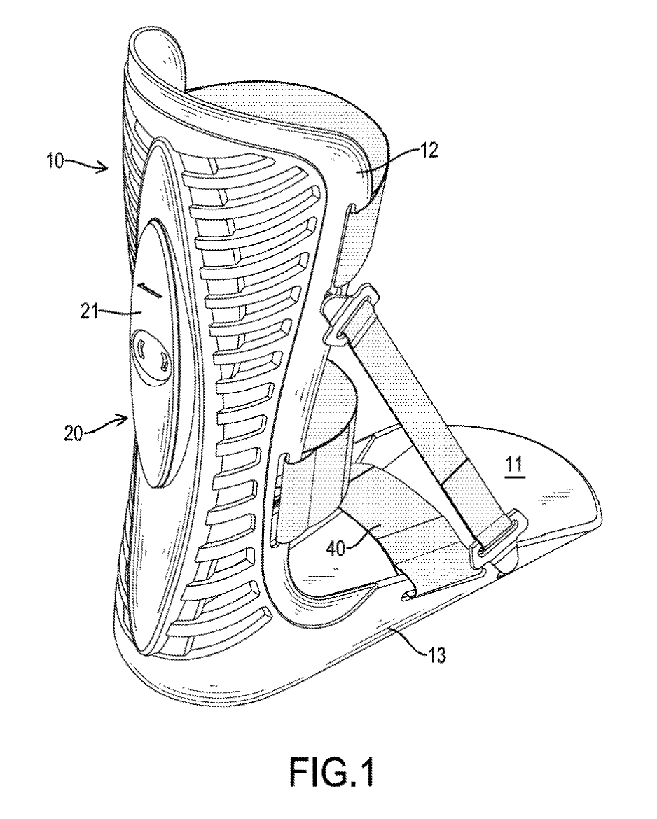

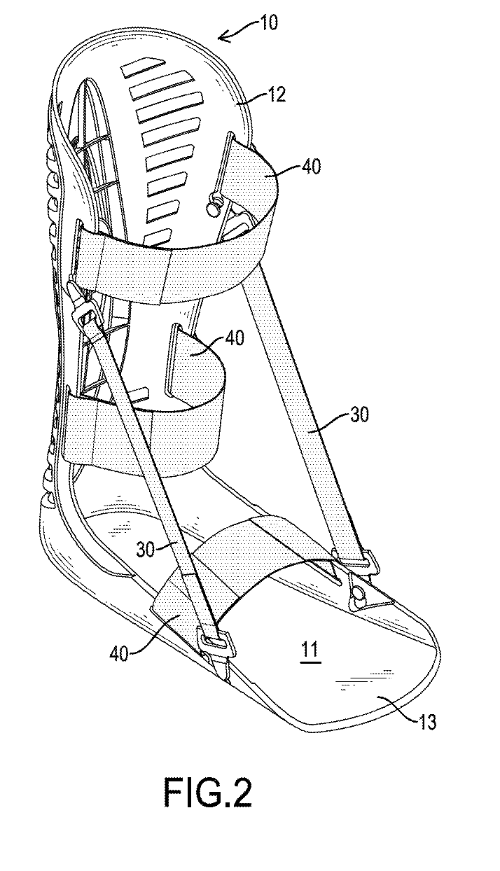

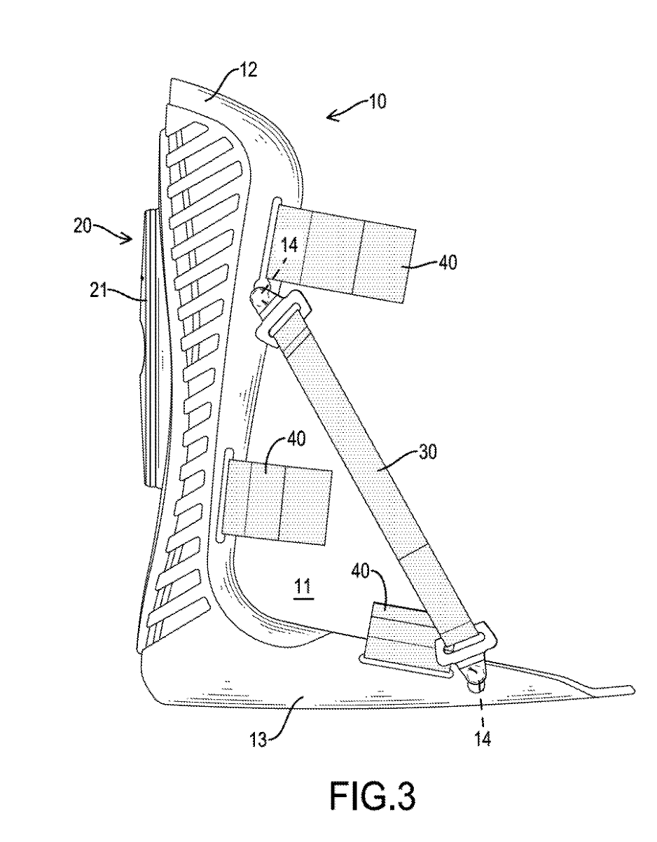

With reference to FIGS. 1 to 5, a preferred embodiment of an ankle stirrup comprises an ankle holder 10, a positioning unit 20, two positioning belts 30, and multiple mounting belts 40.

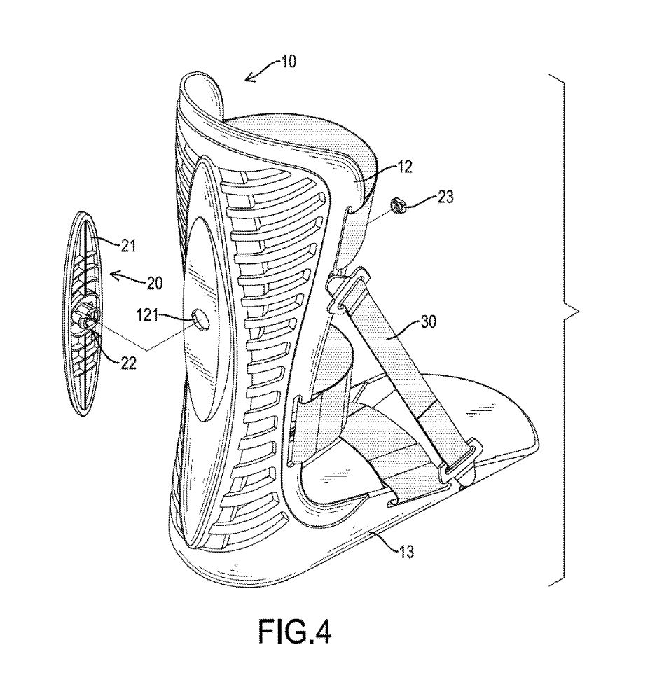

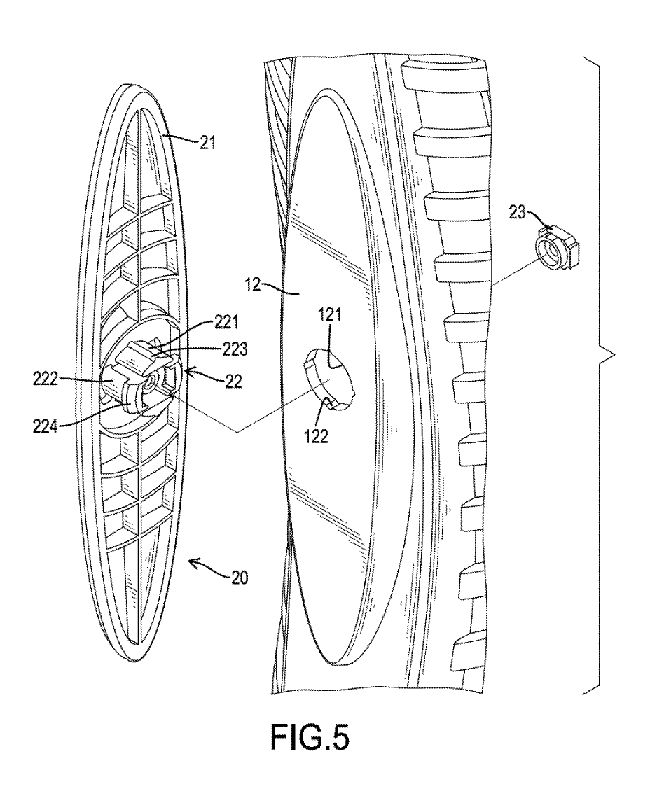

The ankle holder 10 is formed in an L-shape in section, and comprises a holder space 11 formed in a front side of the ankle holder 10. The ankle holder 10 comprises a leg portion 12 and a foot portion 13 connected with the leg portion 12. Alternatively, the leg portion 12 may be two individual parts that are connected with the each other by the positioning belts 30. In use, a foot of a user treads on the foot portion 13, and a leg of the user is supported by the leg portion 12. The ankle holder 10 further comprises two pairs of positioning belt holes 14. One pair of the positioning belt holes 14 is formed in a right side of the ankle holder 10, and the other pair of the positioning belt holes 14 is formed in a left side of the ankle holder 10. The positioning belt holes 14 of each pair are respectively formed in the leg portion 12 and the foot portion 13. With reference to FIGS. 4 and 5, the leg portion 12 further comprises a hole 121 formed through the leg portion 12. The hole 121 comprises four recesses 122 respectively located at a top side, a bottom side, a right side, and a left side of the hole 121.

The positioning unit 20 is mounted in the hole 121 of the ankle holder 10, and comprises a body 21, a rotatable portion 22, and a limiting portion 23. The body 21 is elongated and has a longitudinal direction. The rotatable portion 22 is formed on a front side of the body 21 and is inserted through the hole 121. The rotatable portion 22 is rotatable relative to the hole 121 and comprises two first engaging blocks 221, two second engaging blocks 222, two protrusions 223 and two limiting blocks 224. The first engaging blocks 221 and the second engaging blocks 222 are formed on and protrude from the body 21 at intervals, and are inserted through the hole 121. The protrusions 223 are respectively formed on the first engaging blocks 221 and engaged in two of the recesses 122. The limiting blocks 224 are respectively formed on the second engaging blocks 222 and abut the leg portion 12 to keep the body 21 from detaching from the leg portion 12. The limiting portion 23 is detachably mounted among the first engaging blocks 221 and the second engaging blocks 222.

The positioning belts 30 are respectively mounted on the two pairs of positioning belt holes 14 of the ankle holder 10. Each positioning belt 30 has a top end and a bottom end. The top ends of the two positioning belts 30 are respectively connected with one pair of the positioning belt holes 14 which are located at the leg portion 12, and the bottom ends of the two positioning belts 30 are respectively connected with the other pair of the positioning belt holes 14 which are located at the foot portion 13. Therefore, each positioning belt 30 is connected between the leg portion 12 and the foot portion 13. The mounting belts 40 are mounted on the ankle holder 10. Two ends of each mounting belt 40 are respectively mounted on the right side and the left side of the ankle holder 10.

In use, the user wears the ankle stirrup, wherein the foot and the leg of the user are inserted into the holder space 11. The foot of the user corresponds to the foot portion 13 in position, the leg of the user corresponds to the leg portion 12 in position. The mounting belts 40 are applied for keeping the ankle stirrup from detaching from the leg and the foot of the user.

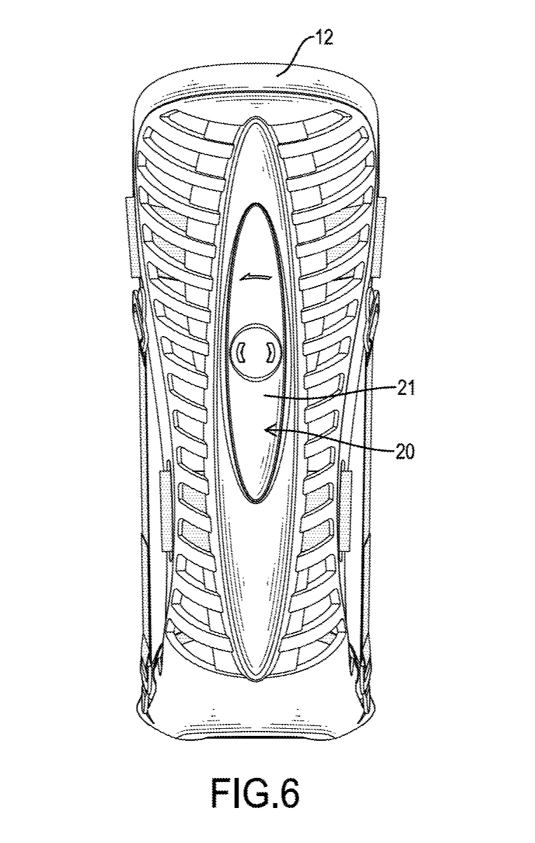

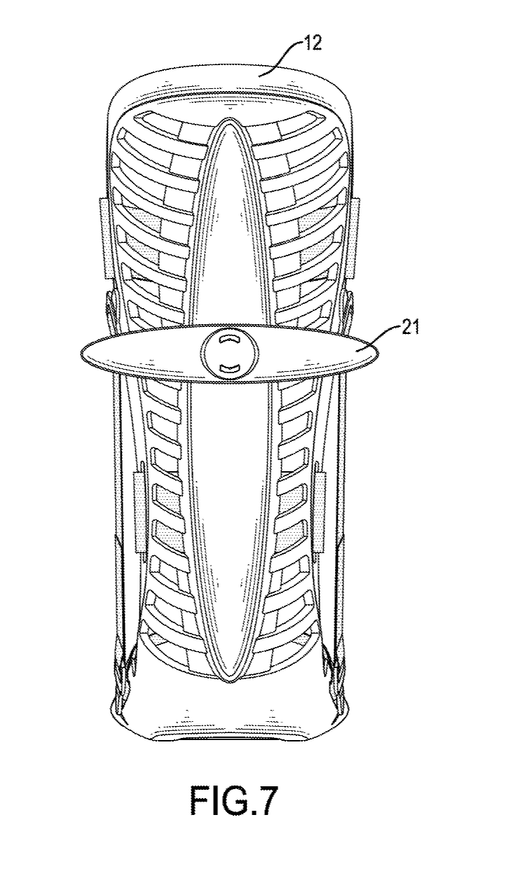

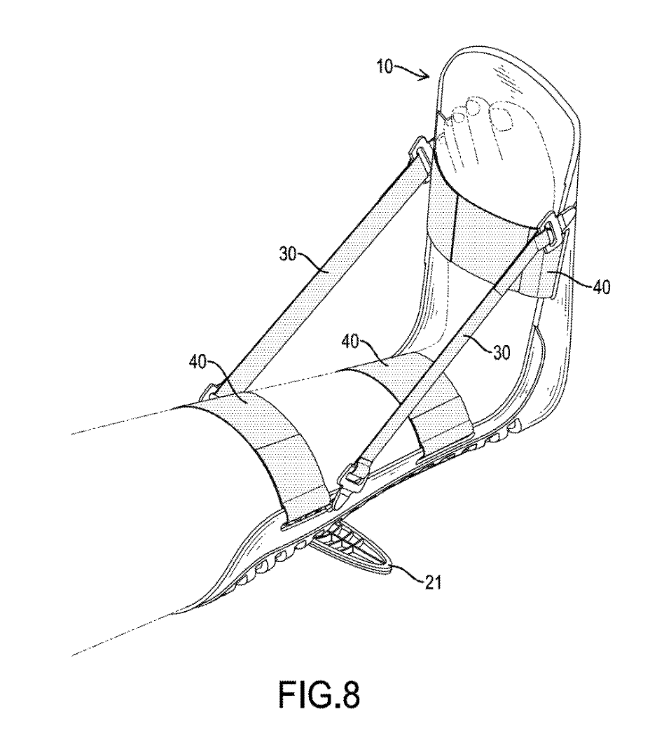

With reference to FIGS. 5 to 8, the positioning unit 20 can be selectively rotatable relative to the leg portion 12 of the ankle holder 10. In a normal situation, the longitudinal direction of the body 21 is parallel with an extension direction of the leg portion 12. The positioning unit 20 can be adjusted to rotate relative to the leg portion 12. First, the limiting portion 23 is detached from the first engaging blocks 221 and the second engaging blocks 222. Then, the body 21 can be rotated by the user. Because the first engaging blocks 221 and the second engaging blocks 222 are formed on the body 21 at intervals and the limiting portion 23 is detached from the first engaging blocks 221 and the second engaging blocks 222, the engaging blocks 221, 222 can be slightly deformed inwardly. When the body 21 is rotated, the protrusions 223 can be detached from the corresponding recesses 122 in which the protrusions 223 are engaged. The body 21 is rotated until the protrusions 223 are engaged in the other two recesses 122, so the longitudinal direction of the body 21 is perpendicular to the extension direction of the leg portion 12. The limiting portion 23 can be mounted among the engaging blocks 221, 222 again to keep the engaging blocks 221, 222 from deforming inwardly. The user can wear the ankle stirrup to lie on a bed, wherein the longitudinal direction of the body 21 is perpendicular to the extension direction of the leg portion 12.

When the body 21 is rotated to be perpendicular to the extension direction of the leg portion 12, the ankle stirrup can be fixed and is not to be turned relative to the bed. Therefore, a fixing effect for an ankle of the user can be improved. Furthermore, each positioning belt 30 is connected between the leg portion 12 and the foot portion 13 to further improve the fixing effect for the ankle of the user.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. An ankle stirrup comprising:
an ankle holder comprising
a foot portion;
a leg portion connected with the foot portion and comprising a hole formed through the leg portion;
a positioning unit mounted in the hole of the ankle holder, and comprising
a body being elongated and having a longitudinal direction;
a rotatable portion formed on a front side of the body, the rotatable portion inserted through, engaged with, and being rotatable relative to the hole; and
a limiting portion detachably mounted in the rotatable portion to keep the rotatable portion from detaching from the hole; and
multiple mounting belts mounted on the ankle holder.

2. The ankle stirrup as claimed in claim 1, wherein the rotatable portion further comprises
at least one first engaging block and at least one second engaging block formed on the front side of the body at intervals and inserted through the hole;
each one of the at least one first engaging block has a protrusion formed on the first engaging block and engaged in the hole; and
each one of the at least one second engaging block has a limiting block formed on the second engaging block and abutting the leg portion to keep the body from detaching from the leg portion.

3. The ankle stirrup as claimed in claim 2, wherein the hole comprises multiple recesses selectively engaged with the at least one protrusion.

4. The ankle stirrup as claimed in claim 1 further comprising two positioning belts respectively mounted on a right side and a left side of the ankle holder, wherein each positioning belt has a top end and a bottom end, and the top ends of the two positioning belts are mounted on the leg portion, and the bottom ends of the two positioning belts are mounted on the foot portion.

5. The ankle stirrup as claimed in claim 2 further comprising two positioning belts respectively mounted on a right side and a left side of the ankle holder, wherein each positioning belt has a top end and a bottom end, and the top ends of the two positioning belts are mounted on the leg portion, and the bottom ends of the two positioning belts are mounted on the foot portion.

6. The ankle stirrup as claimed in claim 3 further comprising two positioning belts respectively mounted on a right side and a left side of the ankle holder, wherein each positioning belt has a top end and a bottom end, and the top ends of the two positioning belts are mounted on the leg portion, and the bottom ends of the two positioning belts are mounted on the foot portion.

7. The ankle stirrup as claimed in claim 4, wherein the ankle holder further comprises two pairs of positioning belt holes, wherein one pair of the positioning belt holes is formed in the right side of the ankle holder, and the other pair of the positioning belt holes is formed in the left side of the ankle holder, and the positioning belts are respectively connected with the two pairs of positioning belt holes.

8. The ankle stirrup as claimed in claim 5, wherein the ankle holder further comprises two pairs of positioning belt holes, wherein one pair of the positioning belt holes is formed in the right side of the ankle holder, and the other pair of the positioning belt holes is formed in the left side of the ankle holder, and the positioning belts are respectively connected with the two pairs of positioning belt holes.

9. The ankle stirrup as claimed in claim 6, wherein the ankle holder further comprises two pairs of positioning belt holes, wherein one pair of the positioning belt holes is formed in the right side of the ankle holder, and the other pair of the positioning belt holes is formed in the left side of the ankle holder, and the positioning belts are respectively connected with the two pairs of positioning belt holes.
